(19) United States
(12) Patent Application Publication
(10) Pub. No.: US 2026/0077211 A1
(43) Pub. Date: Mar. 19, 2026

(54) Phototherapy Using Triple-Polarized Light

(71) Applicant: Erchonia Corporation, LLC, Fountain Inn, SC (US)

(72) Inventors: Travis SAMMONS, Fountain Inn, SC (US); Steven C. SHANKS, Fountain Inn, SC (US); Kevin B. TUCEK, Fountain Inn, SC (US)

(73) Assignee: Erchonia Corporation, LLC, Fountain Inn, SC (US)

(21) Appl. No.: 19/309,121

(22) Filed: Aug. 25, 2025

Related U.S. Application Data
(60) Provisional application No. 63/695,725, filed on Sep. 17, 2024.

(51) Int. Cl. A61N 5/067; A61N 5/06; A61N 5/073

(52) U.S. Cl. CPC A61N 5/067; A61N 2005/0644; A61N 2005/0652; A61N 2005/0659; A61N 2005/0663; A61N 2005/0664; A61N 2005/073

(57) ABSTRACT

A phototherapy device emitting light polarized in different directions is used to treat a patient. Preferably light beams polarized in three directions are applied simultaneously to a desired treatment area on the patient, namely linear polarized light, right-handed circular polarized light, and left-handed circular polarized light.

### CROSS-REFERENCE TO RELATED APPLICATIONS

This application claims the benefit of co-pending U.S. Provisional Application No. 63/695,725 filed Sep. 17, 2024.

### FIELD OF INVENTION

This invention relates generally to the non-invasive application of light therapy. This invention relates particularly to phototherapy using triple-polarized light.

### BACKGROUND

Light therapy has been shown through numerous clinical studies and regulatory clearances to be a safe and effective, simple, non-invasive and side-effect free alternative to medication and surgical procedures for the reduction of symptoms in a variety of conditions. Phototherapy reduces edema, improves wound healing, and relieves pain of various etiologies. It is also used in the treatment and repair of injured muscles and tendons. When the light therapy uses lasers for the light energy, the therapy is known as low-level laser therapy (“LLLT”). Application of LLLT has been shown to have the potential to alter cellular metabolism to produce a beneficial clinical effect such as fat reduction and improved cognitive function.

Mitochondria are a pivotal organelle in cellular healing. Mitochondria provides adenosine triphosphate (ATP), the primary energy currency of cells. Specifically, the mitochondrial electron transport chain, comprising four major protein complexes, is capable of absorbing specific parameters of light. When LLLT is applied to mitochondria, the interaction between photons and these protein complexes induces photoexcitation, facilitating the transfer of electrons through the complexes and creating a proton gradient. This gradient propels the production of ATP via oxidative phosphorylation.

Proteins complexes are complicated molecules. Protein complexes are composed of individual proteins, which are themselves comprised of amino acids linked together by linear peptide bonds. The backbone of an amino acid consists of a central alpha carbon bonded to an amino group, a carboxyl group, a hydrogen molecule, and a side chain. The amino acid molecule is chiral, enabling the existence of L (left-handed) and D (right-handed) stereoisomers. The chirality of amino acids is crucial for protein structure and function, and the majority of naturally occurring amino acids are L-amino acids.

Polarization is a property of light waves which specifies the geometrical direction of the oscillations. Most light sources are not polarized, so the electric field vectors vibrate in many directions, all perpendicular to the direction of propagation. In contrast, in polarized light the electric field vectors vibrate in a single plane. Linear polarized light can be polarized at any angle, whether vertical, horizontal or other angle, as long as the plane is perpendicular to the direction of propagation of the light wave. Light can also be circularly polarized, in which the electric field vectors of the wave have a constant magnitude and rotate at a constant rate in a plane perpendicular to the direction of the wave. Light that emanates from the source in a counterclockwise helix is known as having right-handed circular polarity, and the electric field is rotating clockwise as seen by an observer towards whom the wave is moving. Light that emanates from the source in a clockwise helix is known as having left-handed circular polarity, and the electric field is rotating counter clockwise as seen by an observer towards whom the wave is moving.

Biological molecules may exhibit a preference for one polarization over the other, depending on their specific structure and environment. The alignment of a molecule's linear, straight-chain, achiral or chiral nature with the polarization of incident light can facilitate electronic transitions, altering the molecule's energy state.

Therefore, it is an object to provide devices and methods for treating a patient with phototherapy using light polarized in more than one direction.

### SUMMARY OF THE INVENTION

A phototherapy device emitting light polarized in different directions is used to treat a patient. Preferably light beams polarized in three directions are applied simultaneously to a desired treatment area on the patient, namely linear polarized light, right-handed circular polarized light, and left-handed circular polarized light.

### BRIEF DESCRIPTION OF THE DRAWINGS

FIG. 1A illustrates vertical linear polarized light.

FIG. 1B illustrates left-handed circular polarized light.

FIG. 1C illustrates right-handed circular polarized light.

FIG. 2 is a front perspective view of a hand-held laser device emitting linear polarized light, left-handed circular polarized light, and left-handed circular polarized light simultaneously.

FIG. 3 is a schematic illustration of the optical system of a phototherapy device using light sources emitting light through a dedicated set of optical elements.

FIG. 4 is a schematic illustration of the optical system of a phototherapy device using arrays of light sources emitting light through a shared set of optical elements.

FIG. 5 illustrates treating a patient in an anterior area of stubborn fat with light oriented in three different polarities at the same time.

### DETAILED DESCRIPTION OF THE INVENTION

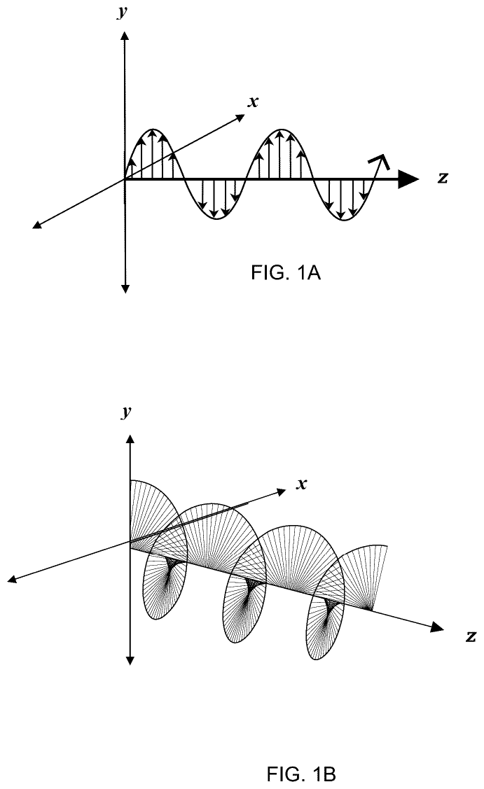

A patient 8 is treated with phototherapy using light polarized in more than one direction, and preferably in three directions. Three directions of polarized light are shown in FIGS. 1A-C. FIG. 1A shows linear polarization 11 in the vertical direction. FIG. 1B shows left-handed circular 14 polarized light. FIG. 1C illustrates right-handed circular 11 polarized light. Other directions of polarized light include linear polarized light in the horizontal direction, linear polarized light at a non-right angle to the direction of light propagation, and elliptically polarized light that is non-circular.

Various optical systems can be employed to polarize the emitted light and shape the beam spot, such as optical elements including lenses, wave plates, filters, mirrors or prisms. Other optical systems include spatial light modulators, digital micromirror device arrays, and acousto-optic modulation. In an exemplary embodiment, polarized light is obtained with optical elements. To obtain linear polarized light, light from a light source emitting non-polarized light is passed through a half-wave plate. Commercially-available laser diodes inherently emit linear polarized light, however, so a half-wave plate is not necessary. The optical systems are typically housed in a housing 21, which may be sized and shaped to be held by a practitioner's hand. Alternatively the optical systems may be distributed outside a housing.

To obtain circular polarized light, the light emitted from the light source as non-polarized light is passed through a half-wave plate and then a quarter wave plate. Light that is inherently emitted as linear polarized light, such as that from a laser diode, is passed through a quarter-wave plate to obtain circular polarized light. For devices in which both right-and left-handed polarized light is desired, one beam of linear polarized light is passed through a first quarter-wave plate to convert it to right-handed circular polarized light and a different beam of linear polarized light is passed through a second quarter-wave plate to convert it to left-handed circular polarized light.

To shape the beam spot of the light that impinges the treated tissue, the light is passed through an optical lens. The optical lens may cause the beam of light to take on various cross-sections such as a round or elliptical beam spot. In a preferred embodiment, the optical lens shapes the polarized emitted light into a line.

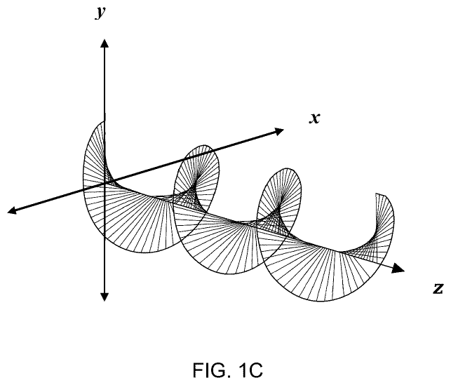

A phototherapy device of the present invention uses one or more light sources. The phototherapy device simultaneously emits linear polarized light 12, right-handed circular polarized light 11, and left-handed circular polarized light 14. The light may be emitted from a hand-held device, a hands-free stand, or a robotic scanner that is aligned over a patient and left unattended. FIG. 2 is a front perspective view of a hand-held phototherapy device 20 in which at least three light sources are housed.

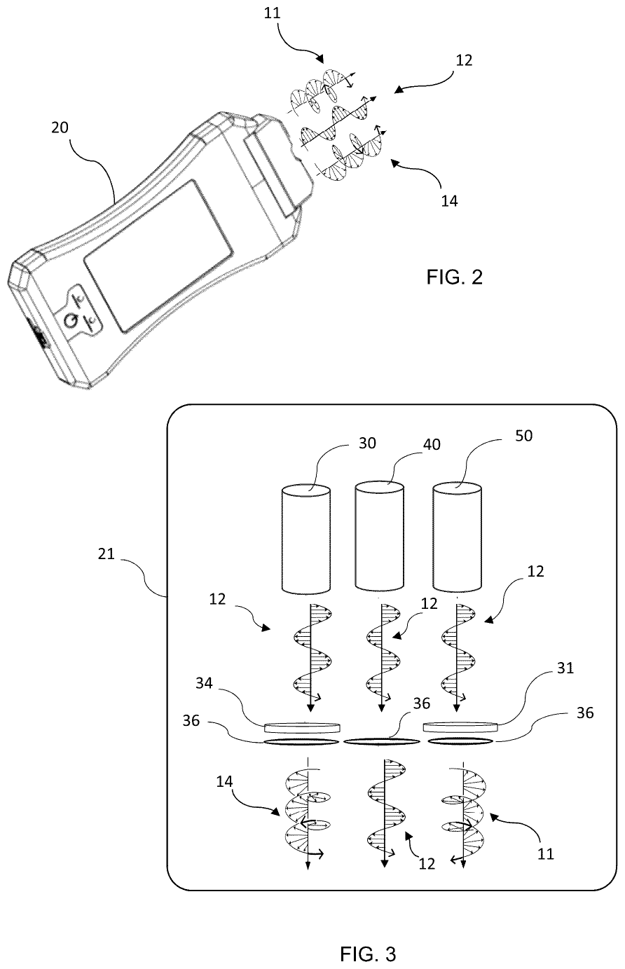

The light emitted from a light source may pass through a dedicated set of optical elements or through a shared set of optical elements. FIG. 3 illustrates the optical system to obtain triple polarized light, with the light from each single light source passing through a dedicated set of optical elements. A laser diode 30 emits linear polarized light 12, which passes through a left quarter-wave plate 34, and optical lens 36, resulting in left-circular polarized light 14. A different laser diode 40 emits linear polarized light 12, which passes through an optical lens 36, resulting in linear polarized light 12. A laser diode 50 emits linear polarized light 12, which passes through a right quarter-wave plate 31, and optical lens 36, resulting in right-circular polarized light 11.

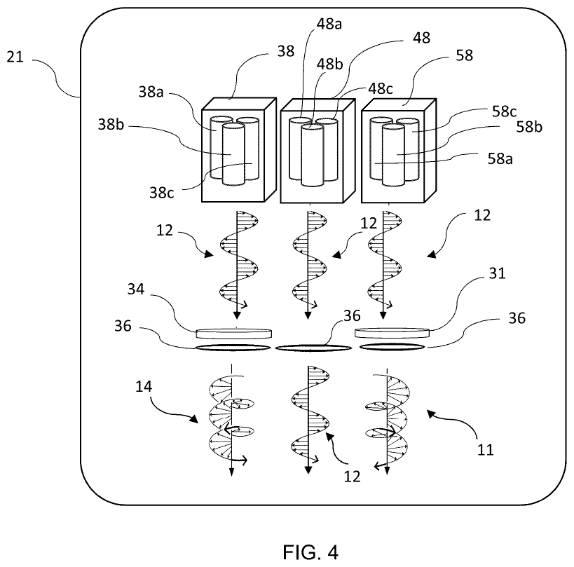

FIG. 4 illustrates another embodiment in which the optical system comprise arrays of light sources. Multiple light sources are housed in array modules 38, 48, and 58 and the light emitted from any one of the light sources in the array passes through a dedicated set of optical elements. Array 38 comprises three light sources 38a, 38b, 38c. The light 12 emitted from each of these light sources 38a, 38b, 38c passes through a left quarter-wave plate 34, and optical lens 36, resulting in left-circular polarized light 14. Similarly, an array 48 comprises three light sources 48a, 48b, 48c, and the light from each passes through an optical lens 36, resulting in linear polarized light 12. Another array 58 comprises three light sources 58a, 58b, 58c. The light 12 emitted from each of these light sources 58a, 58b, 58c passes through a right quarter-wave plate 31, and optical lens 36, resulting in right-circular polarized light 11.

Each light sources emits one or more visible and infrared wavelengths in the range of 400-1000 nm. Each light source may have the same or have a different wavelength. The light sources are typically battery powered, but also be mains powered. The light sources are low power to prevent thermal effects in the tissue, with the output power range of each light source about 1 mW to 1 W. Consequently, the tissue impinged by the light is not heated and is not damaged. Because the tissue impinged by the light is not heated, no mechanism to cool the tissue is needed. The light is applied directly to the patient's tissue in the treatment area with no intervening temperature-reducing elements between the light-emitting device and the treatment area. The applied light energy may be applied continuously or applied with a pulse frequency or frequencies from 0 to 100,000 Hz, where a pulse frequency of zero is referred to as a constant wave.

The three beams of polarized light are shown in FIGS. 2-4 as being arranged in order of left-circular, linear, and right-circular directions of polarization. However, the linear-polarized beam does not need to be between the circular-polarized beams. Instead the beams may be arranged in alternative orders of polarization, such as linear, left-circular, and right-circular; linear, right-circular, and left-circular; right-circular, linear, and left-circular; left-circular, right-circular and linear; right-circular, left-circular and linear; etc. More than three light sources may be used, with some sources having similar polarizations and some sources having different polarizations.

In a preferred embodiment the light sources are semiconductor laser diodes 30. In another embodiment, light-emitting diodes (LEDs) are used as the light sources. Each light source may contain multiple wavelengths (e.g., 635, 520, 405, 450 nm) that emit light independently from one another based on the activation of the signal from software that controls the emissions.

The phototherapy treatments produce photochemical changes of complex biological structures (cells, proteins, nucleic acids, lipids, etc.) via photoexcitation of straight-chain molecules, other achiral molecules, and chiral molecules. These complex structures are involved in producing therapeutic benefits such as RNA and DNA synthesis, increased production of metabolic energy in the form of adenosine triphosphate (ATP) through enhanced mitochondrial protein complex activity. ATP serves as the primary energy source in all biological systems, therefore, the treatment may be applied to, but is not limited to, diseases associated with the musculoskeletal system, wound healing, inflammation, fat loss, skin, and neurological conditions. When the triple polarization is present, the molecule is exposed to a wider range of polarization states, leading to more efficient excitation.

Circular polarized light is preferentially absorbed by helical molecules and linear polarized light preferentially absorbed by molecules with 180 degree bond angles (linear). Helical molecules, such as certain nucleic acids and proteins, often exhibit a specific handedness, classified as L-molecules (left-handed) or D-molecules (right-handed). Nucleic acids, including DNA, are typically right-handed, with B-DNA being the most prevalent form. However, the less common Z-DNA, crucial for gene regulation and DNA repair, possesses a left-handed helical structure. Mitochondrial protein complexes responsible for proton gradient and ATP synthesis, are composed of amino acid chains exhibiting left-handed chirality. The effect of right-handed or left-handed circular polarization on a double DNA-like helix, considered as a whole, is strongly influenced by left-circular polarized light and minimally interacts with right-circular polarized light. Additionally, levodopa (L-DOPA) molecules have been shown to preferentially absorb right-circular polarized light, while D-DOPA molecules favor left-circular polarized light. Analogously, molecules with linear bonds such as amino acids preferentially absorb linear polarized light.

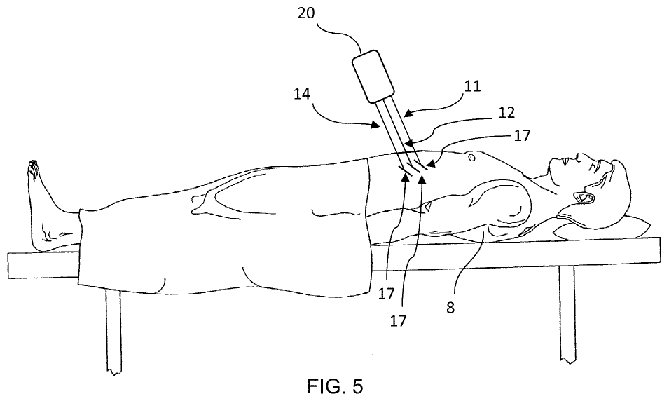

FIG. 5 shows a patient 8 being treated with a hand-held device 20 that is emitting linear polarized light 12, right-handed circular polarized light 11, and left-handed circular polarized light 14 simultaneously. Each of the three beams of light are shaped into lines 17. Emitted light is waved across a person's tissue in the desired treatment area in a continuous, sweeping motion.

Handheld light therapy devices have complete self-contained functionality, control and power. Handheld light therapy devices include one or more light sources, a battery for power, control circuitry, and a control panel such as a touchscreen or keyboard, all enclosed in a relatively small housing sized and shaped to be held by a practitioner's hand. In hands-free devices, the light sources are typically battery powered, but also be mains powered. Whether emitted from a hand-held device, a hands-free stand, or a robotic scanner, preferably the emitted light is passed across a person's tissue in the desired treatment area in a continuous, sweeping motion.

Treatment examples for shoulder pain include:
Example 1: A linear polarized beam of light, a right-circular polarized beam of light, and a left-circular polarized beam of light, each with a wavelength of 640 nm, are simultaneously applied to a patient's shoulder for 10 minutes.
Example 2: A linear polarized beam of light, a right-circular polarized beam of light, and a left-circular polarized beam of light, each with a wavelength of 520 nm, along with a linear polarized beam of light, a right-circular polarized beam of light, and a left-circular polarized beam of light, each with a wavelength of 405 nm, are simultaneously applied to a patient's shoulder for 5 minutes.
Example 3: A linear polarized beam of light, a right-circular polarized beam of light, and a left-circular polarized beam of light, each with a wavelength of 640 nm, are simultaneously applied over the nerve root (C5-C6) corresponding to the shoulder for 2 minutes, followed by simultaneously applying a linear polarized beam of light, a right-circular polarized beam of light, and a left-circular polarized beam of light, each with a wavelength of 405 nm, to the shoulder for 5 minutes.

Treatment examples for low back pain include:
Example 4: A linear polarized beam of light, a right-circular polarized beam of light, and a left-circular polarized beam of light, each with a wavelength of 640 nm are simultaneously applied to a patient's right lumbar region for 10 minutes, during the same time a linear polarized beam of light, a right-circular polarized beam of light, and a left-circular polarized beam of light, all with a wavelength of 520 nm, are simultaneously applied to the patient's left lumbar region. After the 10 minutes of treatment the light application swaps areas such as the 640 nm is used on the left lumbar region, and the 520 nm is used on the right lumbar region.
Example 5: A linear polarized beam of light, a right-circular polarized beam of light, and a left-circular polarized beam of light, each with a wavelength of 640 nm, and a linear polarized beam of light, a right-circular polarized beam of light, and a left-circular polarized beam of light, each all with a wavelength of 405 nm are simultaneously applied for 5 minutes over a patient's center low back area, followed by 5 minutes over the patient's right hip flexor, followed by 5 minutes over the patient's left hip flexor.

Treatment examples for noninvasive fat loss include:
Example 6: A linear polarized beam of light, a right-circular polarized beam of light, and a left-circular polarized beam, each with a wavelength of 520 nm, is applied to a patient's anterior area of stubborn fat for 15 minutes anterior. See FIG. 4. This treatment is followed by applying the same light to the patient's posterior area for 15 minutes. Additional areas with stubborn fat can be treated simultaneously using another set of triple polarized beams, such as right flank and left flank.

Treatment examples for autism include:
Example 7: A device emits 405 nm, 520 nm, and 640 nm wavelengths from three light sources. A 405 nm right circular polarized beam, a 520 nm left circular polarized beam, and a 640 nm linear polarized beam are applied simultaneously over the forehead for 5 minutes.
Example 8: A device with a large laser array. Each array contains 3 separate diode wavelengths (635 nm, 520 nm, 405 nm) that run independently based on the signal from the device software. The light from any laser in the array can be transmitted through a linear lens, one with quarter-wave plate emitting right circular light, and one with quarter-wave plate emitting left circular light. The laser energy is applied for 2 minutes to the patient's forehead and temporal area in a slow sweeping motion, with 635 nm emitting from the linear optical set, 520 nm from right circular optical set, and 405 nm from left circular optical set. This is followed by 2 minutes of 635 nm light emitting from left circular optical set, 520 nm from linear optical set, and 405 nm from right circular optical set. This is followed by 2 minutes of 635 nm emitting from right circular optical set, 520 nm from left circular optical set, and 405 nm from linear optical set.

1. A phototherapy device for treating a patient comprising:
a. an optical system comprising a light source, wherein the optical system is configured to simultaneously emit a linear polarized light beam, a right-handed circular polarized light beam, and a left-handed circular polarized light beam.

2. The phototherapy device of claim 1 wherein the light source comprises one or more laser diodes.

3. The phototherapy device of claim 2 wherein the optical system further comprises:
a. a first quarter-wave plate configured to receive the light emitted from the light source and convert it into right-handed circular polarized light; and
b. a second quarter-wave plate configured to receive the light emitted from the light source and convert it into left-handed circular polarized light.

4. The phototherapy device of claim 1 wherein the optical system further comprises one or more optical elements configured to shape the emitted linear polarized light beam into a first line, the emitted right-handed circular polarized light beam into a second line, and the emitted left-handed circular polarized light beam light into a third line.

5. The phototherapy device of claim 1 wherein the light emitted from the device is applied to the patient's tissue and the light causes no detectable temperature rise of the treated tissue.

6. The phototherapy device of claim 1 wherein the light emitted from the device is applied in a continuous sweeping motion to the patient.

7. The phototherapy device of claim 1 wherein the light source emits one or more wavelengths in the range of 400-1000 nm.

8. A phototherapy device comprising:
a. a housing; and
b. an optical system disposed within a housing, the optical system comprising a light source;
wherein the optical system is configured to simultaneously emit a linear polarized light beam, a right-handed circular polarized light beam, and a left-handed circular polarized light beam from the housing.

9. The phototherapy device of claim 8 wherein the housing is configured to be hand-held.

10. The phototherapy device of claim 8 wherein the light emitted from the device is applied to a patient's tissue and the light causes no detectable temperature rise of the treated tissue.

11. The phototherapy device of claim 8 wherein the light emitted from the device is applied in a continuous sweeping motion to a patient.

12. A hand-held phototherapy device comprising:
a. a housing; and
b. an optical system disposed within the housing, the optical system comprising a first light source, a second light source, and a third light source;
c. wherein the optical system is configured to simultaneously emit:
i. left-handed circular polarized light from the first light source;
ii. linear polarized light from the second light source; and
iii. right-handed circular polarized light from the third light source.

13. The hand-held phototherapy device of claim 12 wherein the optical system further comprises one or more optical elements configured to shape the emitted light into one or more lines.

14. The hand-held phototherapy device of claim 12 wherein the first light source, the second light source, and the third light source are laser diodes.

15. The hand-held phototherapy device of claim 12 wherein the first light source is a first array of laser diodes, the second light source is a second array of laser diodes, and the third light source is a third array of laser diodes.

16. The hand-held phototherapy device of claim 12 wherein the first, second and third light sources emit one or more wavelengths in the range of 400-1000 nm.

17. The hand-held phototherapy device of claim 12 wherein the wavelength of light emitted from the first light source may be the same as or different from the wavelength of light emitted from the second light source or the third light source.

18. The hand-held phototherapy device of claim 12 wherein the light emitted from the device is applied to a patient's tissue and the light causes no detectable temperature rise of the treated tissue.

19. The hand-held phototherapy device of claim 12 wherein the light emitted from the device is applied in a continuous sweeping motion to a patient.

20. The hand-held phototherapy device of claim 12 further comprising:
a. a first quarter-wave plate through which light emitted from the first light source passes to convert it to left-handed circular polarized light; and
b. a second quarter-wave plate through which light emitted from the third light source passes to convert it to right-handed circular polarized light.
